(19) United States
(12) Patent Application Publication
(10) Pub. No.: US 2023/0190777 A1
(43) Pub. Date: Jun. 22, 2023

(54) MAMMALIAN MILK OLIGOSACCHARIDES PREVENT VIRAL INFECTION OF HUMAN EPITHELIUM

(71) Applicant: The Regents of the University of California, Oakland, CA (US)

(72) Inventors: Ishita M. SHAH, Roseville, CA (US); David A. MILLS, Davis, CA (US); J. Bruce GERMAN, Davis, CA (US); Xi CHEN, Davis, CA (US)

(21) Appl. No.: 17/928,397

(22) PCT Filed: Jun. 2, 2021
(86) PCT No.: PCT/US2021/035446

§ 371 Date: Nov. 29, 2022

Related U.S. Application Data
(60) Provisional application No. 63/034,315, filed on Jun. 3, 2020.

(51) Int. Cl. A61K 31/702; A61K 35/20; A61P 31/14; A61P 11/00

(52) U.S. Cl. CPC A61K 31/702; A61K 35/20; A61P 11/00; A61P 31/14

(57) ABSTRACT

Methods and compositions for use of human milk oligosaccharides for prevention and treatment of airway viral infections are provided.

### CROSS-REFERENCE TO RELATED PATENT APPLICATIONS

The present application claims benefit of priority to US Provisional Pat. Application No. 63/034,315, filed Jun. 3, 2020, which is incorporated herein by reference for all purposes.

### BACKGROUND OF THE INVENTION

Milk oligosaccharides (MOs) are present in milk for nourishment and protection, with prebiotic, anti-inflammatory, immunomodulating activities. Certain human MOs that are commercially available are added as safe molecules to infant formula.

### BRIEF SUMMARY OF THE INVENTION

In some embodiments, a nasal spray, nasal drops, an oral spray, an oral rinse, a diffuser, a mist, an inhaler (e.g., metered dose inhaler), a nebulizer or a lozenge comprising one or more milk oligosaccharide is provided. In some embodiments, the one or more milk oligosaccharides are in a concentration sufficient to prevent or inhibit influenza virus or severe acute respiratory syndrome coronavirus 2 (SARS-CoV-2) infection by cells contacted with the one or more milk oligosaccharides. In some embodiments, the one or more milk oligosaccharides comprise 3′-sialyllactose (3′-SL), 2′-Fucosyllactose (2′-FL), 6′-sialyllactose (6′-SL), Lacto-N-neotetraose (LNnT) or a combination thereof.

Also provided is a method of improving barrier function of lung epithelial cells in an animal. In some embodiments, the method comprises administering to the animal the one or more milk oligosaccharides from the nasal spray, nasal drops, an oral spray, an oral rinse, a diffuser, a mist, an inhaler, a nebulizer or a lozenge. In some embodiments the animal is a human. In some embodiments, the one or more milk oligosaccharides comprise 3′-sialyllactose (3′-SL), 2′-Fucosyllactose (2′-FL), 6′-sialyllactose (6′-SL), Lacto-N-neotetraose (LNnT) or a combination thereof.

In some embodiments, the administering comprises administering the one or more milk oligosaccharides via inhalation as delivered by the inhaler. In some embodiments, the administering comprises administering the one or more milk oligosaccharides in the form of a lozenge e.g., in some embodiments, the one or more milk oligosaccharides are dissolved and aerosolized in a lozenge, for administration via inhalation.

In some embodiments, the method comprises treating or preventing infection by a virus of an airway cell in an animal. In some embodiments, the method comprises treating or preventing one or more symptom of asthma, COPD or seasonal allergies in the animal.

Also provided is a method of treating or preventing infection by a virus, bacterium or a fungus of a cell in an animal. In some embodiments, the method comprises contacting the cell with a sufficient amount of one or more milk oligosaccharides to treat or prevent infection by the virus, bacterium or a fungus. In some embodiments, the virus is influenza virus or severe acute respiratory syndrome coronavirus 2 (SARS-CoV-2).

Also provided is a composition comprising one or more milk oligosaccharides, wherein the composition is configured for administration to an animal by at least one of a nasal spray, nasal drops, an oral spray, an oral rinse, a diffuser, a mist, an inhaler (e.g., metered dose inhaler), a nebulizer, or a lozenge.

Also provided are one or more milk oligosaccharides for use in the treatment and/or prevention of an infection by a virus, bacterium or a fungus in an animal.

Also provided are one or more milk oligosaccharides for use in improving barrier function of epithelial cells in an animal.

Also provided are one or more milk oligosaccharides for use in the treatment and/or prevention of asthma, COPD, seasonal allergies, or any combination thereof, in an animal.

### BRIEF DESCRIPTION OF THE DRAWINGS

FIG. 1: A plot showing the effect of infecting MK2-LLC cells (monkey kidney epithelial cells) with Tulane virus (TV), in the absence or presence of pre-incubation with an HMO (2′-FL, LNFP I, or 3′-SL).

FIG. 2: A plot showing the effect of infecting human A549 cells (alveolar epithelial cells) with human coronavirus (HCoV-229E), in the absence or presence of pre-incubation with an HMO (2′-FL or 3′-SL).

FIG. 3: A plot showing the effect of infecting human A549 cells (alveolar epithelial cells) with human coronavirus (HCoV-229E), in the absence or presence of an HMO (2′-FL or 3′-SL). The HMO was either added at the same time as HCoV-229E, or two hours post HCoV-229E addition.

FIG. 4: A plot showing the effect on the cell viability of human A549 cells (alveolar epithelial cells) when infected with human coronavirus (HCoV-229E), in the absence or presence of an HMO (2′-FL or 3′-SL).

FIG. 5: A plot showing the epithelial membrane integrity of monolayers of human A549 cells (alveolar epithelial cells) in the absence or presence of an HMO (2′-FL, 3′-SL, 6′-SL, or LNnT).

FIG. 6: A plot investigating the host-cell anti-viral response of human A549 cells (alveolar epithelial cells) when infected with human coronavirus (HCoV-229E), in the absence or presence of an HMO (2′-FL or 3′-SL).

### DEFINITIONS

A “therapeutic dose” or “therapeutically effective amount” or “effective amount” as used herein may be an amount of the human milk oligosaccharide that prevents, alleviates, abates, or reduces the severity of symptoms of a virus, bacterium, fungus, or any combination thereof, in a patient. A “therapeutic dose” or “therapeutically effective amount” or “effective amount” as used herein may be an amount of the human milk oligosaccharide that prevents, alleviates, abates, or reduces a viral infection, bacterial infection, fungal infection, or any combination thereof, in a patient.

The “degree of polymerization” or “DP” of an oligosaccharide refers to the total number of sugar monomer units that are part of a particular oligosaccharide. For example, a tetra galacto-oligosaccharide has a DP of 4, having 3 galactose moieties and one glucose moiety.

The term “human milk oligosaccharides (HMO)” refers generally to a number of complex carbohydrates found in human milk. Some of these oligosaccharides are specific to human milk whereas others are also found in milk from other species such as bovines. Among the monomers of human milk oligosaccharides are D-glucose (Glc), D-galactose (Gal), N-acetylglucosamine (GlcNAc), L-fucose (Fuc), and sialic acid [N-acetylneuraminic acid (NeuAc)]. Elongation may be achieved by attachment of GlcNAc residues linked in (31-3 or p 1-4 linkage to a Gal residue followed by further addition of Gal in a P-1-3 or P-1-4 bond. Most HMOs carry lactose at their reducing end. From these monomers, a large number of core structures may be formed. Further variations may occur due to the attachment of lactosamine, Fuc, and/or NeuAc. See, e.g., Kunz, C. et al., Annual. Rev. Nutri. (2000) 20:699-722, for a further description of HMOs.

The term “isolated,” when applied to an oligosaccharide, denotes that the oligosaccharide is essentially free of other milk components with which it is associated in the natural state, i.e., in human breast milk. It can be in, for example, a dry or aqueous solution.

The term “purified” denotes that an oligosaccharide has been separated at least in part from other components of human breast milk. Particular oligosaccharides can be purified individually, or a combination of oligosaccharides can be purified away from at least one other component of milk. In some embodiments, the oligosaccharide can be at least 85% pure, optionally at least 95% pure, and optionally at least 99% pure.

The term “barrier function”, in terms of the present disclosure, describes when an epithelial cell barrier functions as desired i.e. it allows the passage of desired molecules e.g. water, gases, solutes, etc. across the epithelial barrier and/or it limits or completely blocks the passage of potentially harmful substances (e.g. antigens, pathogens) across the epithelial barrier.

The expression “improving barrier function”, in terms of the present disclosure, may refer to an improvement and/or increase in epithelial barrier integrity. Epithelial barrier integrity may be quantified by the method detailed in the Examples, or any suitable method for quantifying epithelial barrier integrity known in the art. “Improving barrier function”, in terms of the present disclosure, may refer to an improvement and/or increase in epithelial barrier function and improved cell viability. Epithelial barrier and cell viability may be quantified by the method detailed in the Examples, or any suitable method for quantifying epithelial barrier function and cell viability known in the art.

### DETAILED DESCRIPTION OF THE INVENTION

The inventors have discovered that mammalian milk oligosaccharides improve lung epithelial barrier function and dampen inflammation, providing a protective measure that extends to protect against: pathogen activity e.g., (viral, bacterial, fungal invasion, replication, expansion), and is predicted to lower particulate and pathogen-mediated inflammation associated with pathogen infection or particulate-driven inflammatory responses (e.g., triggers for allergy and asthma, for instance). As discussed below, select human milk oligosaccharides (HMOs), when incubated with host-epithelial cells, prevent virus-mediated cell death as observed by alteration in the numbers of remaining adherent cells. Without intending to limit the scope of the invention, it is believed that the anti-viral effect is due, at least in part, to the milk oligosaccharides mimicking host-cell receptors. Accordingly, methods and compositions for delivery of milk oligosaccharides to airway cells of an animal are provided. The methods can be used preventatively, e.g., before viral infection to reduce or inhibit viral infection upon exposure, or as a treatment, e.g., to prevent or limit infection of additional cells in the animal and/or to reduce the negative effects and thus symptoms caused by the virus. In some embodiments, the methods can be used to achieve lowering of viral infectious load in the upper respiratory tract.

Respiratory infections typically occur when airborne pathogens come into contact with mucous membranes (e.g., nasal membranes, oral membranes, membranes of the throat, etc.) via inhaled aerosol droplets. The barrier function of airway epithelium prevents the spread of infection by intercellular tight and adherens junctions, which regulate epithelial paracellular permeability. However, pathogens are able to subvert the natural barrier function of mucosal membranes and lead to a variety of respiratory infections. In the complex multistep process of viral entry, HMOs play diverse roles in interfering with the process. HMOs can: act as soluble decoy receptors in specific examples like rotavirus (direct binding to host-cells disrupted); bind to glycoproteins and prevent viral binding; mimic histo-blood group antigens (HBGAs); bind to both GI and GII HBGA pockets (norovirus) (Etzold, S. and L. Bode, Curr Opin Virol, 2014. 7: p. 101-7; Hester, S.N., et al., Br JNutr, 2013. 110(7): p. 1233-42); and/or, improve barrier function (Natividad, J.M., et al., Nutrients, 2020. 12(10)) which is significantly disrupted during viral infections (LeMessurier, K.S., et al., Front Immunol, 2020. 11: p. 3.). It is believed the method and compositions can be used to prevent or treat any respiratory pathogen infection, e.g., infection of upper or lower respiratory tracts, or both. The compositions are believed to be effective in maintaining or improving tissue barrier function, preventing or reducing introduction of pathogen into or past the epithelial cells. Exemplary respiratory pathogens can be viral, bacterial or fungal. Exemplary viral infections include but are not limited to those caused by influenza virus (e.g., influenza (flu) viruses A and B), coronaviruses (including but not limited to the SARS-CoV-2 virus or the Middle East respiratory syndrome (MERS) virus), respiratory syncytial virus (RSV), adenoviruses, rhinoviruses or human metapneumovirus. Severe acute respiratory syndrome coronavirus 2 or “SARS-CoV-2” is a virus strain that causes coronavirus disease 2019 (COVID-19). See, e.g., Gorbalenya AE, et al. Nature Microbiology. 5 (4): 536-544 (March 2020).

In addition, because MOs improve barrier function, the compositions and methods herein can also be used to reduce uptake allergens in respiratory tract tissues and maintain tissue barriers, and reduce inflammation that may otherwise break down in response allergens or lung diseases such as asthma or chronic obstructive pulmonary disease (COPD). Thus, in addition to treatment or prevention of respiratory viruses, the methods and compositions described herein can also be used to treat or prevent symptoms of seasonal allergies or asthma or COPD, as well as to treat and/or prevent seasonal allergies, asthma, COPD, or any combination thereof.

It is believed any of a number of milk oligosaccharides (e.g., from humans, bovine, or other mammals) can be used according to the methods and compositions described herein. In some embodiments, the oligosaccharides have a degree of polymerization of 2, 3, 4, 5, 6. 7, 8, or more. In some embodiments, the oligosaccharides have one or more fucosyl moiety. In some embodiments, the milk oligosaccharides are one of 3′-sialyllactose (3′-SL) or 2′-Fucosyllactose (2′-FL), or both. In some embodiments, the milk oligosaccharides are one of the human milk oligosaccharides described in U.S. Pat. No, 8,197,872, for example, Lacto-N-tetraoase, Lacto-N-neotetraose, Monofucosyllacto-N-hexaose, Isomeric Fucosylated Lacto-N-hexaose (1), Isomeric Fucosylated Lacto-N-hexaose (2), Isomeric Fucosylated Lacto-N-hexaose (3), Difucosyl-para-lacto-neohexaose, Difucosyl-para-lacto-hexaose, Difucosyllacto-hexaose, Lacto-N-hexaose, Lacto-N-neohexaose, Para-lacto-hexaose, Para-lacto-neohexaose, Lacto-N-fucopentaose I, Lacto-N-fucopentaose II, Lacto-N-fucopentaose III, or Lacto-N-fucopentaose IV. In some embodiments, the milk oligosaccharide is 6′-sialyllactose (6′-SL). In some embodiments, the milk oligosaccharide is 3′-fucosyllactose (3′-FL). In some embodiments, the compositions described herein comprise two, three or more different MOs, for example 2, 3, or more of the oligosaccharides listed above.

The inventors have surprisingly found that the presence of an HMO protects monkey kidney epithelial cells (MK2-LLC cells) from Tulane virus-mediated cell death, and protects human alveolar epithelial cells (A549 cells) from human coronavirus (HCoV-229E) mediated cell death (see Examples 1 to 3). In addition, the inventors surprisingly discovered that the presence of an HMO led to an increase in human alveolar epithelial barrier integrity (see Example 4). Further, the inventors have shown that the presence of an HMO dramatically attenuates the Type I and Type III interferon response of human alveolar epithelial cells, when infected with human coronavirus (HCoV-229E; see Example 5).

The Tulane virus and the human coronaviruses are known to recognize and/or interact with sialic acids (Tan, M., et al., Sci Rep, 2015. 5: p. 11784). It is believed that the observed protective effect, exerted by the HMOs on the epithelial cells when infected with the Tulane virus or the human coronavirus (HCoV-229E) is due, at least in part, to the HMO acting as a soluble decoy receptor for the particular virus and/or binding to glycoproteins on the epithelial cell surface and preventing viral binding. SARS-CoV-2 and influenza viruses are also known to interact with (O-acetylated) forms of sialic acid (Kim, C.H., Int J Mol Sci, 2020. 21(12).). It is therefore hypothesized that HMOs will prevent and/or treat (i.e. reduce the severity of) viral infections, such as SARS-CoV-2 infection and/or influenza virus infection, via a virus receptor decoy mechanism and/or by affecting the viral binding capacity. Interestingly, the influenza virus glycoprotein hemagglutinin, which is found on the surface of influenza viruses, exhibits specificity to a sialic acid molecule linked to galactose by either an α2,6 or an α2,3 linkage. This process facilitates binding of the influenza virus to host-cells. The same linkages are found in the HMOs, 3′-SL and 6′-SL, which suggests that 3′-SL and/or 6′-SL could be useful in the treatment and/or prevention of influenza virus infection, by acting as an influenza virus receptor decoy.

Further, SARS-CoV-2 infection and influenza A infection have both been shown to disrupt the epithelial barrier and have detrimental effects on epithelial barrier function (Deinhardt-Emmer, S., et al., J Virol, 2021. 95(10) and Short, K.R., et al., Eur Respir J, 2016. 47(3): p. 954-66., respectively). As HMOs are absorbed into the peripheral circulation and therefore have the potential to reach all organs, including the lungs, it is hypothesized that HMOs will prevent and/or reduce the severity of viral infections, such as SARS-CoV-2 infection and/or influenza virus infection, via improvement of epithelial barrier function. It is therefore hypothesized that HMOs will be useful in the prevention and/or treatment of a viral infection, in particular SARS-CoV-2 infection and/or influenza virus infection.

Oligosaccharides as described herein can be obtained by any method. In some embodiments, the oligosaccharides can be purified from a natural source, e.g., human milk. In other embodiments, the oligosaccharides can be generated synthetically, e.g., enzymatically or chemically, e.g., by linking monomeric or oligomeric sugars or by cleaving larger oligosaccharides into the desired oligosaccharide.

Milk oligosaccharides can be derived using any of a number of sources and methods known to those of skill in the art. For example, MOs can be purified from human or animal milks using methods known in the art. One such method for extraction of oligosaccharides from pooled human milk entails the centrifugation of milk at 5,000 x g for 30 minutes at 4° C. and fat removal. Ethanol is then added to precipitate proteins. After centrifugation to sediment precipitated protein, the resulting solvent is collected and dried by rotary evaporation. The resulting material is adjusted to the appropriate pH of 6.8 with phosphate buffer and β-galactosidase is added. After incubation, the solution is extracted with chloroform-methanol, and the aqueous layer was collected. Monosaccharides and disaccharides are removed by selective adsorption of HMOs using solid phase extraction with graphitized nonporous carbon cartridges. The retained oligosaccharides can be eluted with water-acetonitrile (60:40) with 0.01% trifluoroacetic acid. (See, e.g., Ward et al., Appl. Environ. Microbiol. (2006), 72: 4497-4499; Gnoth et al., J. Biol. Chem. (2001), 276:34363-34370; Redmond and Packer, Carbohydr. Res., (1999), 319:74-79.) Individual HMOs can be further separated using methods known in the art such as capillary electrophoresis, HPLC (e.g., high-performance anion-exchange chromatography with pulsed amperometric detection; HPAEC-PAD), and thin layer chromatography. See, e.g., Splechtna et al., J. Agricultural and Food Chemistry (2006), 54: 4999-5006.

Alternatively, enzymatic methods can be used to synthesize the HMOs. In general, any oligosaccharide biosynthetic enzyme or catabolic enzyme (with the reaction running in reverse) that converts a substrate into any of the HMO structures (or their intermediates) may be used in the practice of this invention. For example, prebiotic galacto-oligosaccharides have been synthesized from lactose using the β-galactosidase from L. reuteri (see, Splechtna et al., J. Agricultural and Food Chemistry (2006), 54: 4999-5006). The reaction employed is known as transgalactosylation, whereby the enzyme β-galactosidase hydrolyzes lactose, and, instead of transferring the galactose unit to the hydroxyl group of water, the enzyme transfers galactose to another carbohydrate to result in oligosaccharides with a higher degree of polymerization (Vandamme and Soetaert, FEMSMicrobiol. Rev. (1995), 16:163-186). The transgalactosylation reaction can proceed intermolecularly or intramolecularly. Intramolecular or direct galactosyl transfer to D-glucose yields regioisomers of lactose. Through intermolecular transgalactosylation di-, tri-, and tetra saccharides and eventually higher oligosaccharides specific to Bifidobacterium species are produced. A related method utilizes the β-galactosidase of Bifidobacterium bifidum NCIMB 41171 to synthesize prebiotic galacto-oligosaccharides (see, Tzortzis et al., Appl. Micro. and Biotech. (2005), 68:412-416).

Another approach to the synthesis of the carbohydrates as described herein that combines elements of the methods outlined above entails the chemical or enzymatic synthesis of or isolation of oligosaccharide backbones containing Lacto-N-biose, or Lacto-N-neotetraose from non-human mammalian milk sources (e.g., cows, sheep, buffalo, goat, etc.) and enzymatically adding Lacto-N-biose, fucose and sialic acid units as necessary to arrive at the HMO structures of the present invention. For this purpose, a variety of bifidobacterial carbohydrate modifying enzymes, such as those disclosed in PCT Publication WO 2008/033520 can be utilized. Examples of such oligosaccharide modifying enzymes include sialidases, silate O-Acetylesterases, N-Acetylneuraminate lyases, N-acetyl-beta-hexosaminidase, beta-galactosidases, N-acetylmannosamine-6-phosphate 2-epimerases, alpha-L-fucosidases, and fucose dissimilation pathway proteins, among others, which may be used to catalyze a biosynthetic reaction under the appropriate conditions.

Alternatively, conventional chemical methods may be used for the de novo organic synthesis of or conversion of pre-existing oligosaccharides into the HMO structures of the present invention. See, e.g., March’s Advanced Organic Chemistry: Reactions, Mechanisms, and Structure, 5th Edition.

The compositions of the invention can be administered directly to the animal (e.g., human) subject to prevent or inhibit viral infection by administration to airway cells of the animal, e.g., via inhalation. In some embodiments, the compositions can be administered orally and/or nasally.

The compositions can further comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington’s Pharmaceutical Sciences, 17th ed., 1989).

The compositions, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. The formulations of compounds can be presented in unit-dose or multi-dose sealed containers.

Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Concentration can be determined by a skilled clinician. Variables such as weight and medical history of the recipient, as well as potential adverse effects can be considered in choosing the concentration of the active ingredient. In some embodiments, the one or more milk oligosaccharides are in a concentration sufficient to prevent, reduce or inhibit influenza virus or severe acute respiratory syndrome coronavirus 2 (SARS-CoV-2) infection by cells contacted with the one or more milk oligosaccharides. In some embodiments, the concentration of the oligosaccharide(s) in the composition is 0.01% to up to 10% (w/w, w/v, or v/v), e.g., 0.03-10% (w/w, w/v, or v/v). In some embodiments, the concentration of the oligosaccharide(s) in the composition is 0.01% to up to 20% (w/w, w/v, or v/v), e.g., 0.1 to 10% (w/w, w/v, or v/v). In some embodiments, the concentration of the oligosaccharide(s) in the composition may be 0.05% to 15%, 0.1% to 10%, 0.2% to 7.5%, or 0.25% to 5% (w/w, w/v, or v/v).

The composition may comprise one or more milk oligosaccharides in the range of about 0.01 g/L to about 5.0 g/L. Preferably, the composition comprises one or more milk oligosaccharides in the range of about 0.05 g/L to about 4.0 g/L of the composition. More preferably, the composition comprises one or more milk oligosaccharides in the range of about 0.05 g/L to about 2.0 g/L of the composition. Alternatively, the composition may comprise one or more milk oligosaccharides in the range of about 0.01 g/100 kcal to about 2.0 g/100 kcal. Preferably, the composition comprises one or more milk oligosaccharides in the range of about 0.01 g/100 kcal to about 1.5 g/100 kcal.

The daily dosage of the one or more milk oligosaccharides may be varied depending on the requirement of the patient, the severity of the infection, and the particular form of the one or more milk oligosaccharides. The daily dosage of the one or more milk oligosaccharides may be in the range of about 0.05 milligram per day (mg/day) to about 20 grams per day (g/day). Preferably, the daily dosage of the one or more milk oligosaccharides is in the range of about 0.1 mg/day to about 10 g/day. More preferably, the daily dosage of the one or more milk oligosaccharides is in the range of about 0.15 mg/day to about 5 g/day. Even more preferably, the daily dosage of the one or more milk oligosaccharides is in the range of about 0.2 mg/day to about 4 g/day. The dose of the one or more milk oligosaccharides may be in the form of a single daily dosage. Alternatively, the total daily dosage may be administered in portions throughout the day e.g. two portions, three portions, etc.

In some embodiments, the composition is administered in conjunction with a second agent. In some embodiments the second agent is for treating or preventing a viral infection. In some embodiments, the second agent is a peptide for immunomodulation, a peptide having antibacterial activity to reduce secondary infections, an anti-viral microRNA, 9-(1,3-Dihydroxy-2-propoxymethyl) guanine (ganciclovir) or phosphonoformic acid (PFA).

### EXAMPLES

### Example 1

### Impact of Mammalian Milk Oligosaccharides on Viral Infection of Epithelial Cells

Monkey kidney epithelial (MK2-LLC) cells or human alveolar epithelial (A549) cells were seeded in 12-well plates at 1E5 cells per well.

24-hours post incubation, cells were washed with serum-free media and incubated with the particular HMO at 2 mg/ml final concentration.

16-hours post HMO incubation, the particular virus (for MK2-LLC cells this was Tulane virus, at a multiplicity of infection (MOI) of 0.001; for A549 cells this was human coronavirus (HCoV-229E), at an MOI of 0.01) was added and infection was allowed to proceed.

Images of adherent (live) cells were recorded two-days post infection after aspirating and washing cells with PBS. All wells were washed on Day 3 prior to imaging.

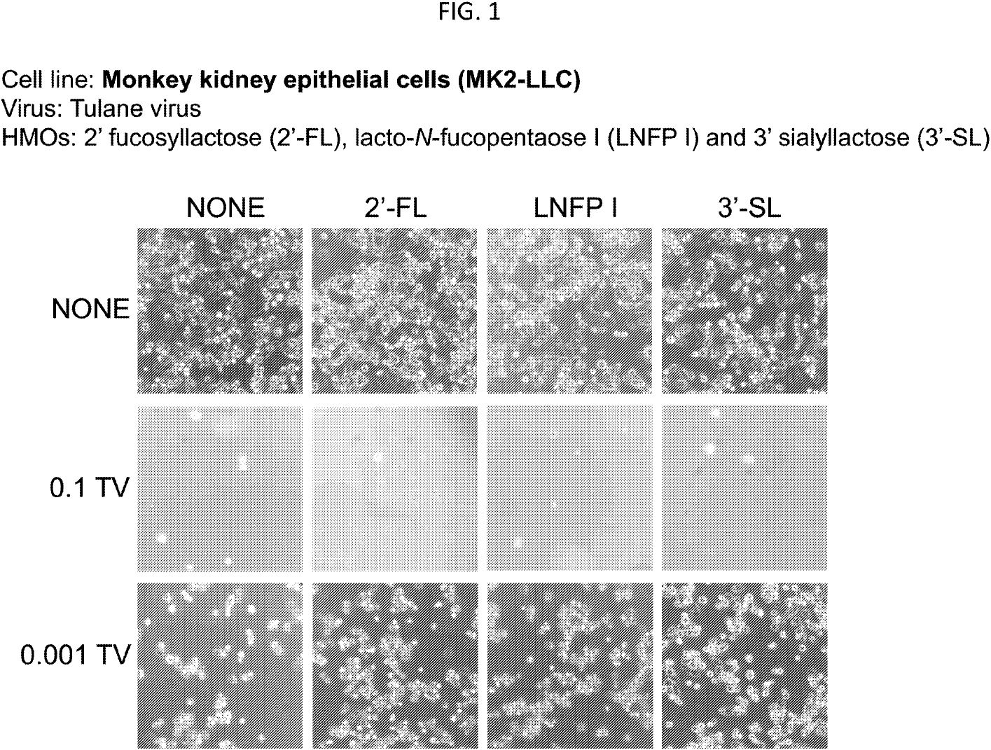

As shown in FIG. 1, the MK2-LLC cells pre-incubated with any of the three HMOs (2′-FL, LNFP I, and 3′-SL) exhibited increased cellular adherence and cell viability in the presence of Tulane virus, when compared to MK2-LLC cells in the absence of an HMO. Analysis of the cellular morphology and cytopathogenic effects indicate that HMO pre-incubation reduces host-cell distress. This suggests that the MK2-LLC cells were protected from Tulane virus-mediated cell death by the presence of an HMO..

It was hypothesized that HMOs enhance cellular adhesion, cell structural integrity and modulate tight junction proteins as a generalized function regardless of the organ from which the epithelial cells are derived, and this function in turn alters the response to pathogenic challenges. With SARS-CoV-2 infections on the rise, it was investigated whether select HMOs had an effect on coronavirus infections in human lung epithelial cells. As proof-of-concept, human alveolar epithelial (A549) cells were incubated in the presence of physiological levels of an HMO (2′-FL or 3′-SL) for 16 hours and infected the cells with a human coronavirus (HCoV-229E).

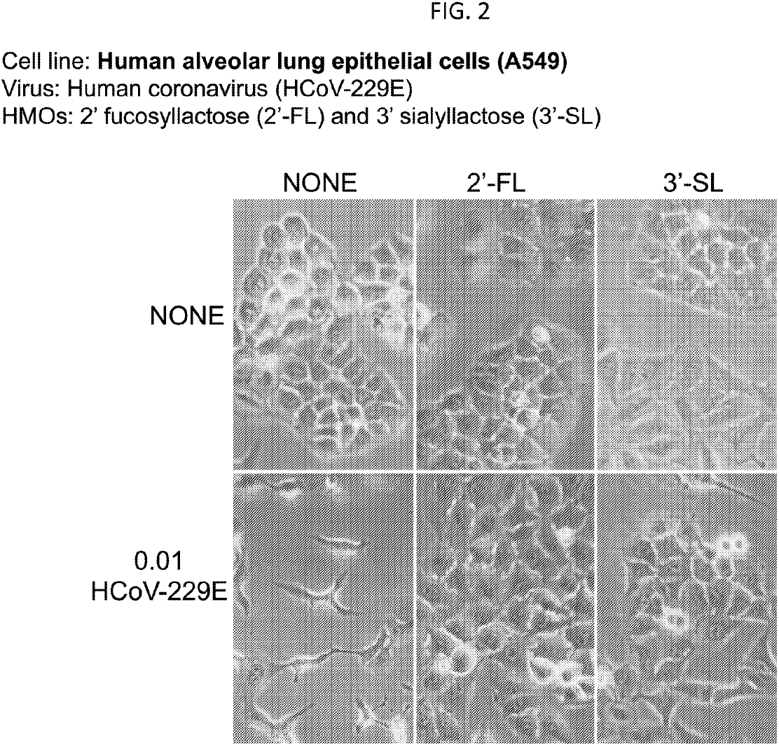

As shown in FIG. 2, dramatic cytopathogenic effects and subsequent loss of adherence of A549 cells was observed when subjected to HCoV-229E infection, in the absence of an HMO. When the A549 cells were pre-incubated with either of the HMOs (2′-FL and 3′-SL), little or no change in A549 epithelial cell morphology was observed up to three days post-infection. This suggests that the A549 cells were protected from human coronavirus-mediated cell death by the presence of an HMO.

### Example 2

### Impact of Milk Oligosaccharides on Viral Infection of Epithelial Cells when Added During or Post-adsorption

Having shown that HMOs exhibit a protective effect on human alveolar epithelial (A549) cells, when infected with human coronavirus (HCoV-229E), it was then investigated whether a similar protective effect would be exhibited when HMOs are added at the same time as the virus, or two-hours post viral addition.

Human alveolar epithelial cells, A549 were seeded in 12-well plates at 1E5 per well.

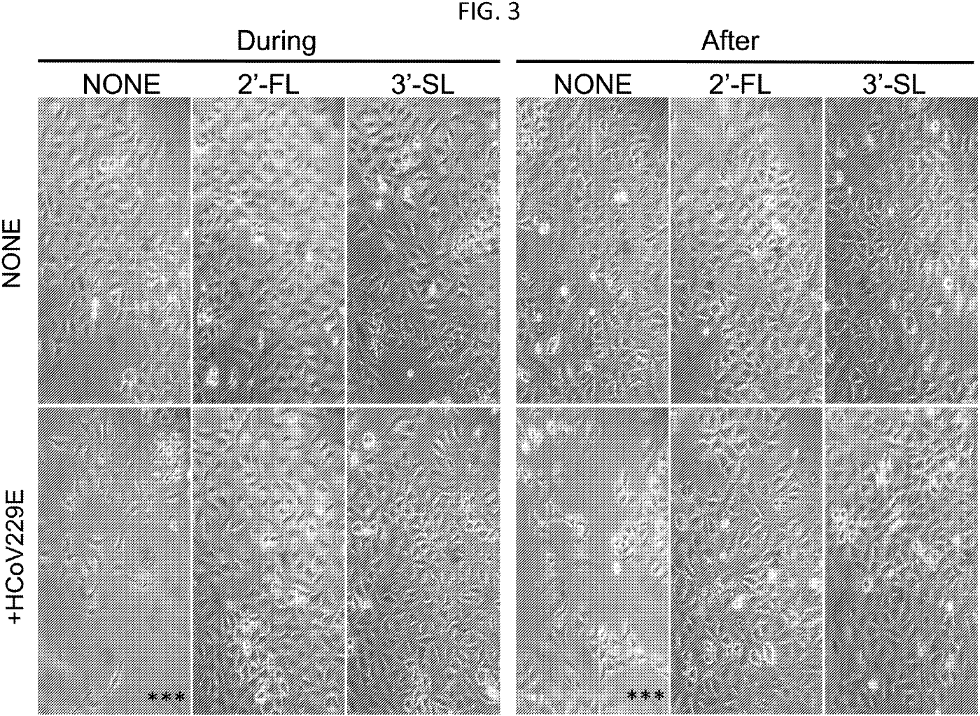

48-hours post incubation (37° C., 5% CO₂), cells were washed with serum-free media (F12/K) and either incubated with 2 mg/ml of the particular HMO (FIG. 3) at final concentration and virus (HCoV-229E, MOI: 0.01) simultaneously (‘During’), or incubated with virus (HCoV-229E, MOI: 0.01) for two hours allowing viral adsorption prior to HMO addition at 2 mg/ml final concentration (‘After’).

Images of adherent (live cells) were recorded three-days post infection after aspirating and washing the cells with PBS.

FIG. 3 shows virus-mediated loss of adherent cells in the absence of an HMO, and cytopathogenic effects. The presence of either of the HMOs (2′-FL and 3′-SL) during infection with virus, or two-hours post viral adsorption reduced the loss of adherent cells, the virus-mediated loss of cell viability and the cytopathogenic effects.

This suggests that even post adsorption, the presence of these HMOs serves to protect the host-cells and control the spread of infection through the epithelial monolayers.

### Example 3

### Quantitative Impact of Viral Infection in the Presence and Absence of Milk Oligosaccharides on Epithelial Host-cell Viability

A549 cells were seeded in a 96-well plate (volume: 150 µl, seeding density: lE5/ml).

48-hours post incubation (37° C., 5% CO₂), cells were washed with serum-free media (F12/K) and incubated with either 2 mg/ml or 5 mg/ml of the particular HMO for 16 hours prior to addition of the particular virus (HCoV-229E, MOI: 0.01) for 48-hours.

Subsequently, media was aspirated and cells were incubated with Calcein-AM dye for 45 min prior to fluorescence measurements.

Fluorescence units were measured (Excitation: 485/20, Emission: 528/20) using Biotek Synergy 2.

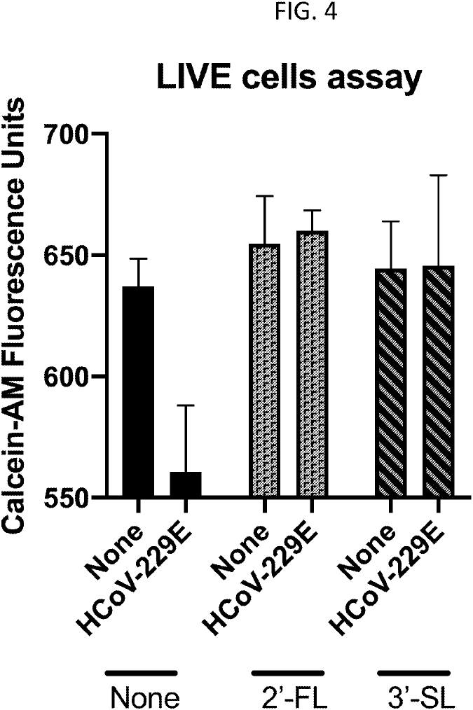

As shown in FIG. 4, a reduction in live cell fluorescence of A549 monolayers was observed in the presence of HCoV-229E, in the absence of an HMO, which indicates a loss of A549 cell viability. In the presence of either of the HMOs (2′-FL and 3′-SL), the A549 cells retain viability even after 48 hours of viral infection. This once again suggests a protective effect of the HMOs.

### Example 4

### Impact of Milk Oligosaccharides on Epithelial Barrier Integrity

A549 cells were seeded in 12 Transwell plates (150 µl volume in the Transwell, apical, seeding density: lE5/ml) with 1 ml media at the base and incubated for 24-hours (37° C., 5% CO₂).

HMOs were added at 2 mg/ml and the Transwell plates were incubated for six hours.

Transepithelial electrical resistance (TEER) was measured to give readings (Ω.cm²) using Millicell ERS volt-ohmmeter for each of the treatments.

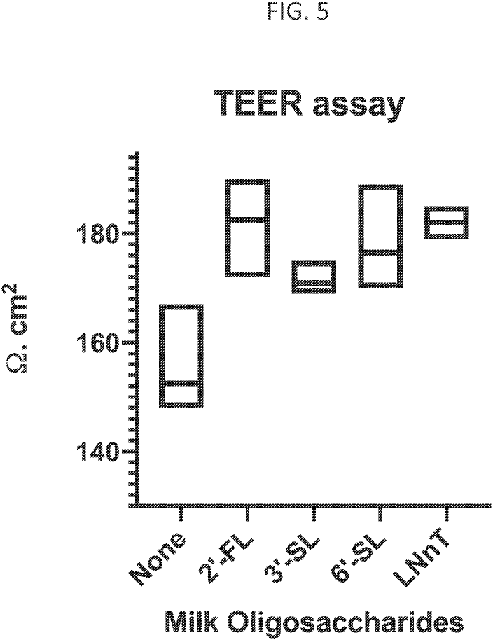

FIG. 5 shows that an improvement in epithelial barrier integrity was observed in the presence of each of the four HMOs six hours post-incubation, when compared to epithelial barrier integrity in the absence of an HMO.

This data suggests that epithelial barrier function improvement, via the presence of the HMOs, could be involved in protection of lung epithelial cells from human coronavirus infection.

### Example 5

### Impact of Milk Oligosaccharides on Host-cell Interferon Response During Viral Infection

Host-cells response to viral infection in the presence and absence of HMOs was measured via expression analysis of interferons, which are known to limit viral replication.

A549 were seeded in 12-well plates at 1E5 per well.

48-hours post incubation (37° C., 5% CO₂), cells were washed with serum-free media (F 12/K) and incubated with 2 mg/ml of the particular HMO at final concentration.

16-hours post HMO incubation, virus was added (HCoV-229E, MOI: 0.01) and the infection was allowed to proceed.

24-hours post infection, media was aspirated and host-cells were subjected to RNA extraction followed by DnaseI treatment and cDNA synthesis to allow for gene-expression measurements using real-time PCR.

Using “None” as reference sample, and β-actin as endogenous control for gene expression, the fold-induction of Type I (IFN-α1, IFN-β1) and Type III interferons (IFN-λ1, IFN-λ2, and IFN-λ3), during HCoV-229E infection of the A549 monolayers, was determined.

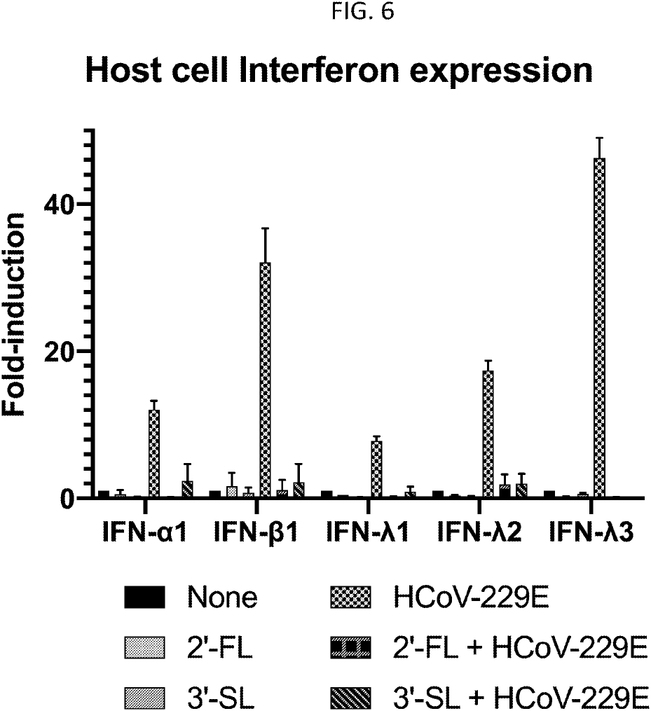

As shown in FIG. 6, both Type I and Type III interferons are induced by the presence of HCoV-229E, and that the response is dramatically attenuated when either of the HMOs (2′-FL and 3′-SL), which once again suggests a protective effect of the HMOs.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A device or product comprising a composition, wherein the device or product is a nasal spray, nasal drops, an oral spray, an oral rinse, a diffuser, a mist, an inhaler (e.g., metered dose inhaler), a nebulizer, or a lozenge, wherein the composition comprises one or more milk oligosaccharides.

2. The device or product of claim 1, wherein the one or more milk oligosaccharides are in a concentration sufficient to prevent, reduce or inhibit influenza virus infection and/or severe acute respiratory syndrome coronavirus 2 (SARS-CoV-2) infection in an animal.

3. The device or product of claim 2, wherein the animal is a human.

4. The device or product of claim 1, wherein the device is a nasal spray.

5. The device or product of claim 1, wherein the product is nasal drops.

6. The device or product of claim 1, wherein the device is an oral spray.

7. The device or product of claim 1, wherein the product is an oral rinse.

8. The device or product of claim 1, wherein the device is a diffuser.

9. The device or product of claim 1, wherein the device is a mist.

10. The device or product of claim 1, wherein the device is an inhaler.

11. The device or product of claim 1, wherein the device is a nebulizer.

12. The device or product of claim 1, wherein the product is a lozenge.

13. The device or product of claim 1, wherein the concentration of the one or more milk oligosaccharides is 0.1% to 10% w/w, 0.1% to 10% w/v, or 0.1% to 10% v/v.

14. The device or product of claim 1, wherein the one or more milk oligosaccharides comprise 3′-sialyllactose (3′-SL), 2′-Fucosyllactose (2′-FL), 6′-sialyllactose (6′-SL), Lacto-N-neotetraose (LNnT), or any combination thereof.

15. A composition comprising one or more milk oligosaccharides, wherein the composition is configured for administration to an animal by at least one of a nasal spray, nasal drops, an oral spray, an oral rinse, a diffuser, a mist, an inhaler (e.g., metered dose inhaler), a nebulizer, or a lozenge.

16. The composition of claim 15, wherein the animal is a human.

17. The composition of claim 15, wherein the wherein the one or more milk oligosaccharides comprise 3′-sialyllactose (3′-SL), 2′-Fucosyllactose (2′-FL), 6′-sialyllactose (6′-SL), Lacto-N-neotetraose (LNnT), or any combination thereof.

18. The composition of claim 15, wherein the concentration of the one or more milk oligosaccharides is 0.01% to 10% w/w, 0.01% to 10% w/v, or 0.01% to 10% v/v.

19-39. (canceled)

40. A method of improving barrier function of lung epithelial cells in an animal, the method comprising administering to the animal the one or more milk oligosaccharides from a nasal spray, nasal drops, an oral spray, an oral rinse, a diffuser, a mist, an inhaler, a nebulizer, a lozenge, or any combination thereof.

41. The method of claim 40, wherein the animal is a human.

42. The method of claim 40, wherein the one or more milk oligosaccharides comprise 3′-sialyllactose (3′-SL), 2′-Fucosyllactose (2′-FL), 6′-sialyllactose (6′-SL), Lacto-N-neotetraose (LNnT), or any combination thereof.

43. The method of claim 40, wherein the administering comprises administering the one or more milk oligosaccharides via inhalation as delivered by the inhaler.

44. The method of claim 40, wherein the administering comprises administering the one or more milk oligosaccharides in the form of a lozenge.

45. The method of claim 40, wherein the method comprises treating or preventing infection by a virus of an airway cell in an animal.

46. The method of claim 40, wherein the method comprises treating or preventing one or more symptom of asthma, COPD or seasonal allergies in the animal.

47. A method of treating or preventing infection by a virus, bacterium or a fungus of a cell in an animal, the method comprising contacting the cell with a sufficient amount of one or more milk oligosaccharides to treat or prevent infection by the virus, bacterium or a fungus.

48. The method of claim 47, wherein the virus is an influenza virus or severe acute respiratory syndrome coronavirus 2 (SARS-CoV-2), or both.
